(19) United States
(12) Patent Application Publication
(10) Pub. No.: US 2025/0035627 A1
(43) Pub. Date: Jan. 30, 2025

(54) METHODS AND RELATED ASPECTS FOR DETECTING AND QUANTIFYING CHIMERIC ANTIGEN RECEPTOR T CELLS IN SAMPLES

(71) Applicants: ARIZONA BOARD OF REGENTS ON BEHALF OF ARIZONA STATE UNIVERSITY, Scottsdale, AZ (US); Mayo Foundation for Medical Education and Research, Rochester, MN (US)

(72) Inventors: Shaopeng WANG, Chandler, AZ (US); Nanxi YU, Tempe, AZ (US); Chao CHEN, Tempe, AZ (US); Xinyu ZHOU, Tempe, AZ (US); Januario E. CASTRO, Phoenix, AZ (US); Eider F. Moreno Cortes, Scottsdale, AZ (US)

(73) Assignees: ARIZONA BOARD OF REGENTS ON BEHALF OF ARIZONA STATE UNIVERSITY, Scottsdale, AZ (US); Mayo Foundation for Medical Education and Research, Rochester, MN (US)

(21) Appl. No.: 18/778,278

(22) Filed: Jul. 19, 2024

Related U.S. Application Data
(60) Provisional application No. 63/528,298, filed on Jul. 21, 2023.

(51) Int. Cl. G01N 33/569; B01L 3/00; C12N 5/00; G01N 33/543; G01N 33/68; G06V 10/70

(52) U.S. Cl. CPC G01N 33/56972; B01L 3/502753; C12N 5/0087; G01N 33/54346; G01N 33/6863; G06V 10/70; B01L 2300/0627; B01L 2300/0681

(57) ABSTRACT

Provided herein are methods of differentiating cell types in a cell population. The methods include removing at least some non-Chimeric Antigen Receptor (CAR)-T cells from a fluidic sample obtained from a subject without centrifuging the fluidic sample to produce a purified fluidic sample. The fluidic sample comprises CAR-T cells and the non-CAR-T cells. The methods also include capturing cells in the purified fluidic sample on a surface that comprises binding moieties that bind at least to the CAR-T cells to produce a captured cell population. In addition, the methods also include distinguishing the CAR-T cells from the non-CAR-T cells in the captured cell population using a trained machine learning model to produce a captured CAR-T cell population data set. Additional methods as well as related devices and systems are also provided.

### CROSS-REFERENCE TO RELATED APPLICATIONS

This application claims priority to, and the benefit of, U.S. Provisional Patent Application Ser. No. 63/528,298, filed Jul. 21, 2023, the disclosure of which is incorporated herein by reference.

### BACKGROUND

Chimeric antigen receptor (CAR) T-cell therapy, which has exhibited impressive outcomes in patients with non-Hodgkin lymphoma over recent years, received FDA approval in 2017. This therapy has shown high efficacy in patients with refractory large B-cell lymphoma after conventional therapy's failure. However, the expression level of CAR-T cells and cytokines in patients' blood is closely linked to the severity of clinical response and adverse events, such as cytokine release syndrome. Up to 86% of patients experienced this symptom, and 67% developed neurotoxicity. About 15% of patients receiving CAR-T cell therapy died of significant toxicity. Standard approaches to monitor the CAR-T cells expansion efficiency are Fluorescence-Activated Cell Sorting (FACS) or Magnetic-Activated Cell Sorting (MACS). However, these methods are time consuming, requiring a professional researcher over 4 hours for a single measurement, excluding sample acquisition and transport time. Hence, patients must be hospitalized for days before receiving their lab results. Thus, timely monitoring of CAR-T cells' expression levels and cytokines in patients' blood can provide patients and doctors with appreciably more prevention and intervention opportunities and help the scientists with a better understanding of the underlying causes of the disease. Home monitoring of the treatment progression by patients can significantly reduce the cost of the therapy.

The process of generating CAR-T cells is straightforward. Briefly, polyclonal T-cells are extracted from the patient body and activated by Interleukin 2 (IL-2) and Cluster of Differentiation 3/Cluster of Differentiation 28 (CD3/CD28) coated micro-beads. This activation step is crucial in T-cell culture since T-cells will not expand and grow efficiently in culture media without it. Next, retrovirus or non-viral vectors are used to insert the targeted genome into T cells, enabling the T cells to express specific anti-target receptors (Chimeric antigen receptor) on their surface membrane. After ex vivo expansion and purification, the engineered T cells with chimeric antigen receptors are injected back to patient blood stream and can directly attack specific cancer cells under the receptors' guidance. Therefore, we can use the CAR-T-specific antigen to capture and count these cells.

Whole blood-based testing requires the removal of Red Blood Cells (RBCs) prior to measurement due to their significant background effects, such as blocking the sensing surface, affecting the quantification of other blood components, and raising the noise-to-signal ratio of results. The current standard approach of CAR-T cell quantification in clinical or research studies is FACS and MACS. Portable home-use devices such as lateral flow strips or microfluidic chips that test cytokine or protein level in fingertip whole blood samples, have not been widely published used as diagnostic devices. These methods require one or more steps to remove RBCs to enrich the target analytes. Classic processes for whole blood sample RBC removal pretreatment include direct centrifugation or Ficoll-Paque/Hypaque, which separates RBCs through density gradient difference, requiring high-speed centrifuge speed up to 11,000 rpm. Another method is using osmotic pressure difference with Ammonium Chloride Potassium (ACK) lysing buffer, which exchanges bicarbonate for chloride through anion exchangers (AE1) (Chloride shift) on the membrane of RBCs, Therefore, the chloride in the lysis buffer will diffuse into the RBCs due to low ion gradient inside while exporting bicarbonate to the outside of the membrane. However, ACK lysing buffer has a low lysing efficiency (˜60%) for RBCs and affects white blood cell viability, which does not meet the requirement of low-density WBCs counting.

Microfluidic devices, such as the application of membrane filters, paper-based filtration, cotton threads, and dielectrophoresis in microfluidic chip analysis, have emerged as a field of blood components separation and miniaturization analysis. Some of them also involved the participation of ACK lysis buffer, combining with magnetic beads or using geometrical channel effects related to blood components bio-physical properties for cells isolation. These microfluidic lab-on-chip designs showed great potential to shorten patient hospitalization time and provide prospective manufacturing ideas with accurate and efficient analysis. However, the microfluidic designs increase the cost and complexity of the assay, limiting further mass production for clinical diagnosis and examination. Others have pointed out that the need for precision manufacturing techniques (photolithography necessity) and the use of specialized materials can be expensive. Furthermore, the fabrication process can be time-consuming and requires specialized equipment, personnel, which adds to the cost.

Accordingly, there is a need for optimal methods for quantifying a particular type of white blood cell, possessing a greater size and lower density compared to red blood cells, within a miniaturized point-of-care apparatus, without necessitating supplementary centrifugation.

### SUMMARY

This present disclosure provides a point-of-care diagnostic system for monitoring chimeric antigen receptor (CAR)-T cell expansion. In some embodiments, the system implements a centrifuge-free RBCs removal method with a label-free imaging-based CAR-T counting device and that can quantify the target CAR-T cells from a drop of blood or other sample type. These and other attributes of the present disclosure will be apparent upon a complete review of the specification, including the accompanying figures.

According to various embodiments, a method of differentiating cell types in a cell population is presented. The method includes removing at least some non-Chimeric Antigen Receptor (CAR)-T cells from a fluidic sample obtained from a subject without centrifuging the fluidic sample to produce a purified fluidic sample, wherein the fluidic sample comprises CAR-T cells and the non-CAR-T cells. The method also includes capturing cells in the purified fluidic sample on a surface that comprises one or more binding moieties that bind at least to the CAR-T cells to produce a captured cell population. In addition, the method also includes distinguishing the CAR-T cells from the non-CAR-T cells in the captured cell population using a trained machine learning model to produce a captured CAR-T cell population data set, thereby differentiating the cell types in the cell population.

Various optional features of the above embodiments include the following. The binding moieties are conjugated to the surface. The binding moieties bind to receptors on the CAR-T cells. The binding moieties comprise one or more anti-CAR-T cell antibodies or antigen binding portions thereof. The binding moieties comprise one or more antigens or functional portions thereof. The antigens comprise recombinant antigens. The antigens comprise CD19 molecules and wherein the CAR-T cells comprise CD19-targeted CAR-T cells. The method comprises counting the CAR-T cells in the captured cell population. The trained machine learning model is configured to distinguish the CAR-T cells from the non-CAR-T cells in the captured cell population based at least in part on one or more morphological characteristics of the CAR-T cells and/or the non-CAR-T cells. The distinguishing step further comprises taking one or more images of the captured cell population bound to the binding moieties using an optical imaging mechanism to produce a captured cell population image data set when the fluidic sample flows through the cavity via the opening, and wherein the trained machine learning model uses the captured cell population image data set to produce the captured CAR-T cell population data set. A cavity of a microfluidic device comprises the surface. The method further comprises quantifying the CAR-T cells in the captured CAR-T cell population data set to produce a quantified CAR-T cell data set. The method comprises administering, or altering an administration of, at least one therapy to the subject based at least in part on the quantified CAR-T cell data set. The method further comprises detecting one or more cytokines present in the fluidic sample. The method comprises detecting the cytokines present in the fluidic sample using gold nanoparticle labeled detection antibodies or antigen binding portions thereof. The method further comprises quantifying the cytokines present in the fluidic sample to produce a quantified cytokine data set. The method comprises administering, or altering an administration of, at least one therapy to the subject based at least in part on the quantified cytokine data set.

Various additional optional features of the above embodiments include the following. The non-CAR-T cells comprise red blood cells (RBCs). The at least some non-CAR-T cells are present in the fluidic sample in a form of agglutinated cells and wherein the removing step comprises filtering the agglutinated cells from the fluidic sample using a filtering mechanism. The filtering mechanism comprises a filter having a pore size of no more than about 10 μm. The fluidic sample has a volume of between about 25 μL and about 75 μL. The fluidic sample is a whole blood sample. The method comprises agglutinating red blood cells (RBCs) in the whole blood sample prior to and/or concurrent with removing the at least some CAR-T cells from the fluidic sample. The method comprises agglutinating the RBCs using at least one anti-blood type antibody.

According to various embodiments, a device is presented. The device comprises a housing structure that comprises a body structure comprising at least one cavity at least partially disposed within the body structure and an opening that fluidly communicates with the cavity. At least one surface of the cavity comprises one or more binding moieties that bind at least to Chimeric Antigen Receptor (CAR)-T cells. The device also comprises a filter mechanism operably connected, or connectable, to the housing structure, which filter mechanism is configured to prevent at least some non-CAR-T cells in a fluidic sample from contacting the surface of the cavity when the fluidic sample flows through the cavity via the opening. The device also includes a detector operably connected, or connectable, to the housing structure, which detector is configured to detect a captured cell population bound to the binding moieties when the fluidic sample flows through the cavity via the opening. In addition, the device also includes a controller operably connected, or connectable, to the housing structure, which controller comprises, or is configured to communicate with, a trained machine learning model that distinguishes the CAR-T cells from the non-CAR-T cells in the captured cell population to produce a captured CAR-T cell population data set when the fluidic sample flows through the cavity via the opening.

According to various embodiments, a system is presented. The system comprises a fluidic sample receiving area in which at least one surface of the fluidic sample receiving area comprises one or more one or more binding moieties that bind at least to receptors on Chimeric Antigen Receptor (CAR)-T cells. The system also comprises a filter mechanism operably connected, or connectable, to the fluidic sample receiving area and/or to another system component, which filter mechanism is configured to prevent at least some non-CAR-T cells in a fluidic sample from contacting the surface of the fluidic sample receiving area when the fluidic sample flows through the fluidic sample receiving area. The system also includes a detector operably connected, or connectable, to the fluidic sample receiving area and/or to another system component, which detector is configured to detect a captured cell population bound to the binding moieties when the fluidic sample flows through the fluidic sample receiving area. In addition, the system also comprises a controller operably connected, or connectable, to the fluidic sample receiving area and/or to another system component, which controller comprises, or is configured to communicate with, a trained machine learning model that distinguishes the CAR-T cells from the non-CAR-T cells in the captured cell population when the fluidic sample flows through the fluidic sample receiving area.

Various optional features of the above embodiments include the following. The filtering mechanism comprises a filter having a pore size of no more than about 10 μm. The binding moieties are conjugated to the surface. The binding moieties bind to receptors on the CAR-T cells. The binding moieties comprise one or more anti-CAR-T cell antibodies or antigen binding portions thereof. The binding moieties comprise one or more antigens or functional portions thereof. The antigens comprise recombinant antigens. The antigens comprise CD19 molecules and wherein the CAR-T cells comprise CD19-targeted CAR-T cells. A cartridge comprises the body structure and wherein the housing structure is configured to reversibly receive the cartridge. The detector comprises an optical imaging mechanism that is configured to take one or more images of the captured cell population bound to the binding moieties to produce a captured cell population image data set when the fluidic sample flows through the cavity via the opening, and wherein the trained machine learning model is configured to use the captured cell population image data set to produce the captured CAR-T cell population data set. The trained machine learning model is configured to distinguish the CAR-T cells from the non-CAR-T cells in the captured cell population based at least in part on one or more morphological characteristics of the CAR-T cells and/or the non-CAR-T cells. The controller is configured to transmit at least a portion of the captured CAR-T cell population data set to a remote device or system.

Various additional optional features of the above embodiments include the following. The controller further comprises, or is configured to further communicate with, one or more non-transient instructions, which when executed by a processor, further perform at least: counting the CAR-T cells in the captured cell population The controller further comprises, or is configured to further communicate with, one or more non-transient instructions, which when executed by a processor, further perform at least: quantifying the CAR-T cells in the captured CAR-T cell population data set to produce a quantified CAR-T cell data set. The controller further comprises, or is configured to further communicate with, one or more non-transient instructions, which when executed by a processor, further perform at least: outputting at least one therapy recommendation based at least in part on the quantified CAR-T cell data set. The detector is further configured to detect one or more cytokines present in the fluidic sample when the fluidic sample flows through the cavity via the opening. The controller further comprises, or is configured to further communicate with, one or more non-transient instructions, which when executed by a processor, further perform at least: quantifying the cytokines present in the fluidic sample to produce a quantified cytokine data set. The controller further comprises, or is configured to further communicate with, one or more non-transient instructions, which when executed by a processor, further perform at least: outputting at least one therapy recommendation based at least in part on the quantified cytokine data set. The filter mechanism is configured to prevent at least some of the non-CAR-T cells present in the fluidic sample in a form of agglutinated cells from contacting the surface of the cavity when the fluidic sample flows through the cavity via the opening. The system comprises a fluid conveyance mechanism operably connected, or connectable, to the housing structure and/or to the cartridge, which fluid conveyance mechanism is configured to flow the fluidic sample through the cavity via the opening. The fluidic sample has volume of between about 25 μL and about 75 μL when the fluidic sample flows through the cavity via the opening. The controller is configured to wirelessly communicate with a computer that comprises the trained machine learning model. The device is hand-held. The device comprises a point-of-care device. A kit comprising the device and/or the cartridge.

### BRIEF DESCRIPTION OF DRAWINGS

FIG. 1 is a flow chart that schematically shows exemplary method steps of differentiating cell types in a cell population according to some aspects disclosed herein.

FIG. 2 is a schematic diagram of an exemplary system suitable for use with certain aspects disclosed herein.

FIG. 3. Workflow of the centrifuge-free rapid optical imaging-based CAR-T cell detection platform. Sample preparation: 1) Collect 50 μL whole blood (spiked with predetermined concentration of Jurkat CAR-T cells for calibration curve generation and testing purpose); 2) Mix the whole blood sample with 25 μL anti-blood type antibody and leave in room temperature to agglutinate for 30 mins; 3) Pass the agglutinated sample through a filter, and collect the fluid through a capillary tube to remove agglutinated RBCs. CAR-T cells Capture: 4) Load filtered samples into the functionalized sensor chips and incubate for 15 minutes to capture CAR-T cells to the sensor surface; 5) Wash unbound cells away with buffer. Data collection and analysis: 6) Take optical microscopy images of the captured cells on the sensor chips surfaces; 7) Process the image to count number of CAR-T cells in the images; 8) Determine CAR-T cell concentration in the blood sample via a calibration curve.

FIGS. 4A-4C. Principle and result of centrifuge-free RBCs removal from whole blood. A) Schematic of agglutinated sample filtration for removing RBCs from whole blood sample. B) compares the membrane filter efficiency between 20 μm and 25 μm membrane filters. The filtering efficiency is the ratio of the white blood cells (WBCs) concentration after passing through the membrane filter and the concentration before passing through the membrane filter (Efficiency=WBC_{after}/WBC_{before}). Data were generated with LUNA II cell counter; C) compares the WBCs' concentration in undiluted whole blood samples (Before filtering) and agglutinated and filtered (25 μm filter) samples. The Hemacytometer assay was applied. The standard deviation of triplicate tests determines the error bars (mean±SD).

FIGS. 5A-5M. Results of CAR+ T cell captured by recombinant CD19 coated surface. Panels A to H compare the images recorded before and after flushing step in different groups. Panels I to L are images taken after the flushing step with the specific Concentration of Jurkat CAR-T cells in spiked whole blood samples. Panel M is the standard curves on CAR-T cell counts vs concentrations. The x-axis represents the CAT-T cell concentrations of whole blood or buffer spiked samples, and the Y-axis represents the cells' number remaining on the surface after the flushing step. Each value consists of 30 images counting information through the entire channel, where the error bars are the standard deviation of at least triplicate experiments (mean±SD). The log-log linear model fits the standard curves (R_{wb}²=0.9895, R_{buffer}²=0.9624). The limit of detection is 1 cell/μL.

FIGS. 6A-6H. Selective capture of Jurkat CAR-19⁺ T cells by recombinant CD19-coated surface with fluorescent staining and flow cytometry verification. Panel 6A to 6F provide brightfield Jurkat CAR-T cells and wild type Jurkat T cells before and after flushing. Panels 6C and 6F are fluorescence images of CD19-PE-stained cells after the flushing step. Panel 6G presents a comparison of the percentage Jurkat CAR19⁺ T cells identified by flow cytometry (77.00±1.68) and our sensors (87.67±5.65) with error bars representing the standard deviation of three or more replicates. Notably, no significant difference was observed between the two methods. In panel 6H are quantification of the fraction of Jurkat CAR-19+T cells attached to the surface, calculated as the ratio of the number of cells attached before and after flushing. Error bars represent the standard deviation of three or more replicates, and significant difference was observed between the two groups (P<0.0001).

FIGS. 7A-7G. Automated algorithm overview and results of algorithm output using spiked whole blood at varying CAR T-cell concentrations. (A) Section of image representing 0.1% of total captured image area. (B) Localized standard deviation filter. (C) Modified match filtering. (D) Negative value thresholding. (E) Identification of local maxima and radial segmentation of original brightfield image. (F) Identification of CAR T-cells using ilastik ML-based software. (G) Output of automated algorithm analyzing whole blood spiked with 4 different concentrations of CAR T-cells with whole blood control.

FIG. 8. Illustration of the CAR-T cell detection setup 1) Flow rate control section containing an automatic syringe pump and tubing system for injecting samples and buffer; 2) Experimental perform section regarding a homemade fluidic chip with one straight channel for sample testing and observing; 3) Image acquisition section combining a classic inverted microscope, a CMOS camera for recording sequence figures, and a mercury light as the fluorescence light source; 4) Data processing applied with Matlab (R2022a) and Fiji Image J for data visualization.

FIG. 9. Schematic figure of the fluidic chip structure. Our homemade fluidic chip contains three layers and four components, which include one 25×50 mm coverslip, one laser-cut channel made from two layers of double-sided tapes, a pre-drilled cover slide with two holes used as the inlet and outlet, and two connection needle supporters made with polydimethylsiloxane (PDMS) for supporting the injection tubes' needles. State channel size.

FIGS. 10A-10F. The surface chemistry of coverslip modification. A) Clean the coverslip with oxygen plasma to introduce hydroxyl groups on the surface. B) Drench the coverslip into 1% (v/v) GPTMS solution to perform epoxy-functionalization via silanization reaction. C) Incubate and immobilize CD19 protein on the pre-modified coverslip through Epoxy-amino binding covalently {Mateo, 2003 #170}. The capturing protein structure is formed. D) Immobilize the target cell through the CD19:CAR-19 binding procedure. E) and F) Apply CD19 PE-conjugated protein for fluorescent staining verification.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and devices, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen: As used herein, the term “antigen” refers is a moiety or molecule that can trigger an immune response upon binding to a specific T-cell receptor or antibody.

Antigen Binding Portion: As used herein, the term “antigen binding portion” refers to a portion of an antibody that specifically binds to a CAR-T cell (e.g., to a receptor displayed on the surface of the CAR-T cell), e.g., a molecule in which one or more immunoglobulin chains is not full length, but which specifically binds to a CAR-T cell. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Binding: As used herein, the term “binding” or “binding interaction”, typically refers to a non-covalent association between or among two or more entities. “Direct” binding involves physical contact between entities or moieties; “indirect” binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell).

Binding Moiety: As used herein, the term “binding moiety” refers to a portion of a chemical compound or structure that selectively or preferentially binds to another chemical compound or structure. In some embodiments, for example, a surface (e.g., a solid surface of a microfluidic cavity or channel) is functionalized or conjugated with binding moieties (e.g., anti-CAR-T cell antibodies or antigen binding portions thereof, or antigens or functional portions thereof), which selectively or preferentially bind to CAR-T cells (e.g., receptor proteins of the CAR-T cells).

Chimeric Antigen Receptor T Cell: As used herein, the term “chimeric antigen receptor T cell” or “CAR-T cell” refers to T cells having receptor proteins that have been engineered to give the cells a new ability to target a specific antigen. The receptor proteins are chimeric in that they typically combine both antigen-binding and T cell activating functions into a single receptor protein. CAR T cells have various therapeutic uses, including the treatment of cancer.

Conjugate: As used herein, “conjugate” refers to a reversible or irreversible connection between two or more substances or components. In some embodiments, for example, gold nanoparticles (AuNPs) are connected to antibodies and/or to antigen binding portions thereof. In some embodiments, binding moieties (e.g., antigens or functional portions thereof, or anti-CAR-T cell antibodies or antigen binding portions thereof) are conjugated to a solid support surface (e.g., a surface disposed in a cavity of a microfluidic cartridge or device). In some embodiments, conjugation is via one or more linker compounds.

Cytokine: As used herein, the term “cytokine” refers to a category of small proteins (typically about 5-25 kDa in size) that are secreted by certain cells of the immune system and involved in cell signaling processes. Examples of cytokines, include interferons, lymphokines, interleukins, chemokines, and tumor necrosis factors.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Detect: As used herein, the term “detect,” “detecting,” or “detection” refers to an act of determining the existence or presence of one or more target analytes (e.g., a CAR-T cell and/or a cytokine in a sample.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

Epitope: As used herein, “epitope” refers to the part of an antigen to which an antibody and/or an antigen binding portion binds.

Functional Portion: As used herein, the phrase “functional portion” in the context of antigens refers to a modified or unmodified portion or fragment of an antigen that retains its ability (e.g., comprises a relevant epitope) to specifically bind to targeted CAR-T cells.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Sample: As used herein, “sample” or “fluidic sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide), which is assayed as described herein. A sample may also be any body fluid or excretion (for example, but not limited to, blood, urine, stool, saliva, tears, bile) that contains cells, cell components, or non-cellular fractions.

Subject: As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

Chimeric antigen receptor (CAR)-T-cell immunotherapy is a rapidly growing treatment for cancer. However, the widespread application of this therapy is limited by significant side effects such as cytokine release syndrome (CRS) and neurological events (NE), which can be triggered by the increasing quantity of engineered CAR-T-cells in the inflammatory triggered environment. Furthermore, approximately half of the patients who receive this immunotherapy show a non-prominent response and require intensive hospitalized administration, monitoring, and follow-up. Therefore, a rapid and economical detection method is urgently needed to estimate the density of functional CAR-T cells in patients' blood at point of care setting. Accordingly, in some aspects, the present disclosure provides a centrifuge-free Rapid Optical Imaging (ROI)-based platform to count CAR T-cells in whole blood samples. In some embodiments, the platform integrates separation, collection, and detection steps in a single microfluidic chip, eliminating the need for centrifugation, staining procedures, or professional interpretation. In some embodiments, for example, after agglutinating red blood cells (RBCs) and passing a membrane filter, a microfluidic channel system is used to separate and collect white blood cells (WBCs) from 50 μL of whole blood samples. In some embodiments, the collected WBCs are then loaded onto a functionalized sensor chip with a CAR-T specific detection zone using capillary-driven force. In some embodiments, the CAR-T cell density in the blood is determined by digitally counting captured cells via optical imaging. This simple imaging-based detection platform can be configured as a point-of-care device for the prognosis of cancer patients treated with CAR-T cellular therapies, among other applications. The platform is rapid, cost-effective, and can improve patient outcomes by enabling the early detection of CAR-T cell density in the blood and the adjustment of treatment strategies accordingly.

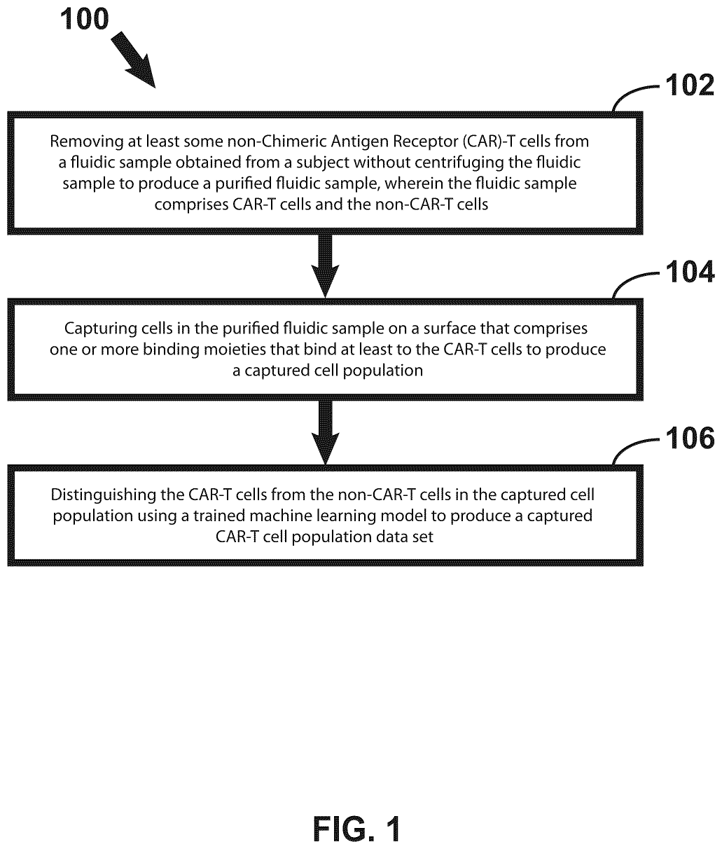

To illustrate, FIG. 1 is a flow chart that schematically shows exemplary method steps of differentiating cell types in a cell population according to some aspects disclosed herein. As shown, method 100 includes removing (or at least separating) at least some non-Chimeric Antigen Receptor (CAR)-T cells from a fluidic sample obtained from a subject without centrifuging the fluidic sample to produce a purified fluidic sample (step 102). The fluidic sample comprises CAR-T cells and the non-CAR-T cells (e.g., red blood cells (RBCs), etc.). Method 100 also includes capturing cells in the purified fluidic sample on a surface (e.g., a surface of a cavity or channel disposed in a microfluidic device or cartridge) that comprises binding moieties (e.g., conjugated to the surface) that bind at least to the CAR-T cells to produce a captured cell population (step 104). Method 100 also includes distinguishing the CAR-T cells from the non-CAR-T cells in the captured cell population using a trained machine learning model to produce a captured CAR-T cell population data set (step 106).

In some embodiments, the binding moieties bind to receptors on the CAR-T cells. In some embodiments, the binding moieties comprise one or more anti-CAR-T cell antibodies or antigen binding portions thereof. In some embodiments, the binding moieties comprise one or more antigens or functional portions thereof. In some embodiments, the antigens comprise recombinant antigens. In some embodiments, the antigens comprise CD19 molecules and the CAR-T cells comprise CD19-targeted CAR-T cells.

In some embodiments, method 100 comprises counting the CAR-T cells in the captured cell population. In some embodiments, the trained machine learning model is configured to distinguish the CAR-T cells from the non-CAR-T cells in the captured cell population based at least in part on one or more morphological characteristics of the CAR-T cells and/or the non-CAR-T cells. Typically, the distinguishing step comprises taking one or more images (e.g., still and/or video images) of the captured cell population bound to the binding moieties using an optical imaging mechanism to produce a captured cell population image data set when the fluidic sample flows through the cavity via the opening. In some embodiments, the trained machine learning model uses the captured cell population image data set to produce the captured CAR-T cell population data set.

In some embodiments, method 100 comprises quantifying the CAR-T cells in the captured CAR-T cell population data set to produce a quantified CAR-T cell data set. In some embodiments, method 100 comprises administering, or altering an administration of, at least one therapy to the subject based at least in part on the quantified CAR-T cell data set. In some embodiments, method 100 further comprises detecting one or more cytokines present in the fluidic sample. In some embodiments, method 100 comprises detecting the cytokines present in the fluidic sample using gold nanoparticle labeled detection antibodies or antigen binding portions thereof. In some embodiments, method 100 comprises quantifying the cytokines present in the fluidic sample to produce a quantified cytokine data set. In some embodiments, method 100 comprises administering, or altering an administration of, at least one therapy to the subject based at least in part on the quantified cytokine data set.

In some embodiments, the non-CAR-T cells comprise red blood cells (RBCs). In some embodiments, at least some non-CAR-T cells are present in the fluidic sample in a form of agglutinated cells. In some of these embodiments, the removing step of method 100 comprises filtering the agglutinated cells from the fluidic sample using a filtering mechanism (e.g., a filter membrane or the like). In some embodiments, the filtering mechanism comprises a filter having a pore size of no more than about 10 μm. In some embodiments, the fluidic sample has a volume of between about 25 μL and about 75 μL (e.g., about 30 μL, about 35 μL, about 40 μL, about 45 μL, about 50 μL, about 55 μL, about 60 μL, about 65 μL, or about 70 μL). In some embodiments, the fluidic sample is a whole blood sample, although other sample types are also optionally utilized. In some embodiments, method 100 comprises agglutinating red blood cells (RBCs) in the whole blood sample prior to and/or concurrent with removing the at least some CAR-T cells from the fluidic sample. In some of these embodiments, for example, method 100 comprises agglutinating the RBCs using at least one anti-blood type antibody.

The present disclosure also provides various devices (e.g., point-of-care microfluidic devices, etc.). In some embodiments, the devices comprise a housing structure that comprises a body structure comprising at least one cavity at least partially disposed within the body structure and an opening (e.g., an inlet port) that fluidly communicates with the cavity. In some embodiments, a cartridge comprises the body structure. In these embodiments, the housing structure is typically configured to reversibly receive the cartridge. Typically, at least one surface of the cavity comprises one or more binding moieties that bind at least to Chimeric Antigen Receptor (CAR)-T cells. In some embodiments, the devices also comprise a filter mechanism operably connected, or connectable, to the housing structure. The filter mechanism is configured to prevent at least some non-CAR-T cells in a fluidic sample from contacting the surface of the cavity when the fluidic sample flows through the cavity via the opening. In some embodiments, the devices also include a detector (e.g., a Rapid Optical Imaging (ROI) as described herein) operably connected, or connectable, to the housing structure (e.g., within sensory communication with the cavity or a microfluidic cartridge that includes the cavity). The detector is configured to detect a captured cell population bound to the binding moieties when the fluidic sample flows through the cavity via the opening. In some embodiments, the devices also include a controller operably connected, or connectable, to the housing structure, which controller comprises, or is configured to communicate with, a trained machine learning model that distinguishes the CAR-T cells from the non-CAR-T cells in the captured cell population to produce a captured CAR-T cell population data set when the fluidic sample flows through the cavity via the opening.

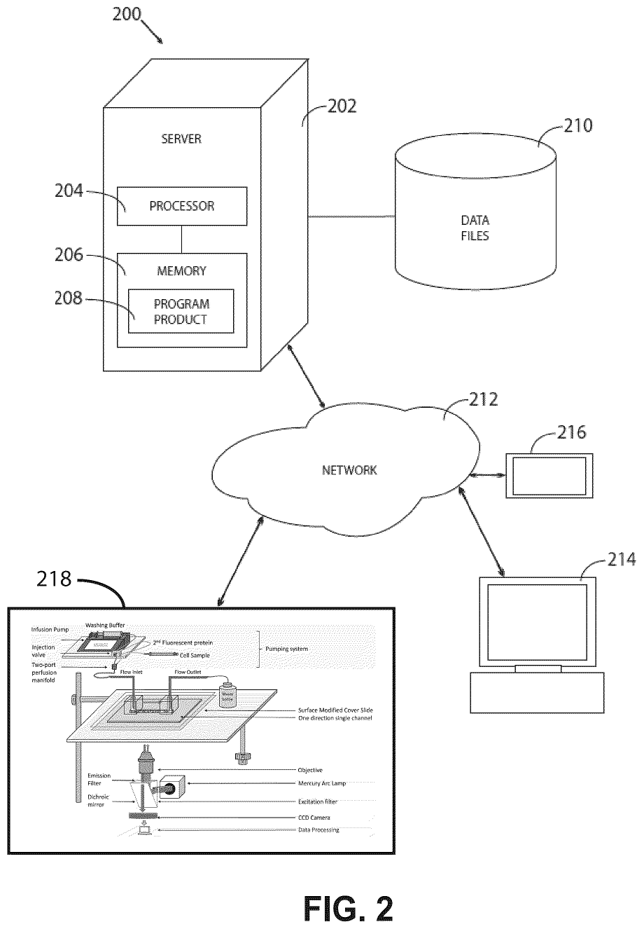

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving imaging data sets or results, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for differentiating cell types in a cell population as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to CAR-T cell counts, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: distinguishing CAR-T cells from non-CAR-T cells in a captured cell population using a trained machine learning model to produce a captured CAR-T cell population data set.

Typically, imaging is obtained using device or subassembly 218. As shown, device or subassembly 218 includes a pumping system for flowing cell samples and other reagents to a channel of a microfluidic device, as described herein. CAR-T cells in the cell samples are counted and distinguished from non-CAR-T cells using the optical imaging system shown, which includes an objective lens and a CCD camera.

### EXAMPLE

### A Centrifuge-Free Rapid Optical Imaging-Based Platform for Point of Care Assessment of CAR T-Cell Expansion Via Machine Learning Verification

### Introduction

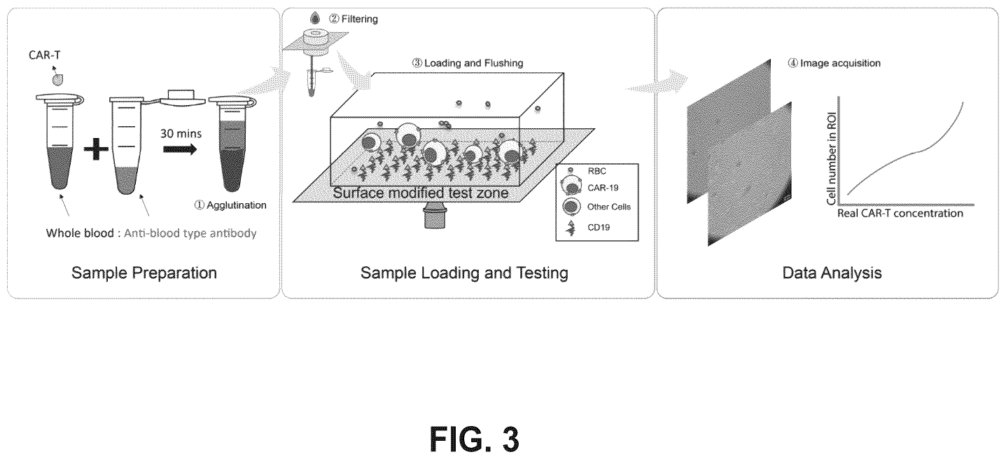

In this example we report a point-of-care diagnostic system ideal for monitoring chimeric antigen receptor (CAR)-T cells' expansion. The system integrates a novel centrifuge-free RBCs removal method with a label-free imaging-based CAR-T counting device and can quantify the target CAR-T cells from a drop of blood. The workflow of the assay is shown in FIG. 3. Major steps include sample preparation, CAR-T cell capture, and image collection and processing. First, we remove red blood cell (RBC) interference from whole blood samples without using centrifugation or RBCs disruption procedure. Whole blood is mixed with an RBCs agglutination antibody solution for 30 minutes to allow RBCs to aggregate, and then the aggregated RBCs are filtered out by a hydrophilic polycarbonate membrane filter with a pore size larger than single cells. Next, the filtered blood is loaded in a sensor chip functionalized with CD19 protein to capture the corresponding CAR-T cells with CD19 receptors expressed on cell surface. After 15 mins incubation, non-specific attached cells are wash out, and the remaining cells are imaged and counted. The result is cross validated with fluorescence imaging after incubating fluorescent labeled CD19 proteins with the captured cells, confirming these cells are CD19 positive CAR-T cells.

### Materials and Methods

### Sample Delivery and Detection System

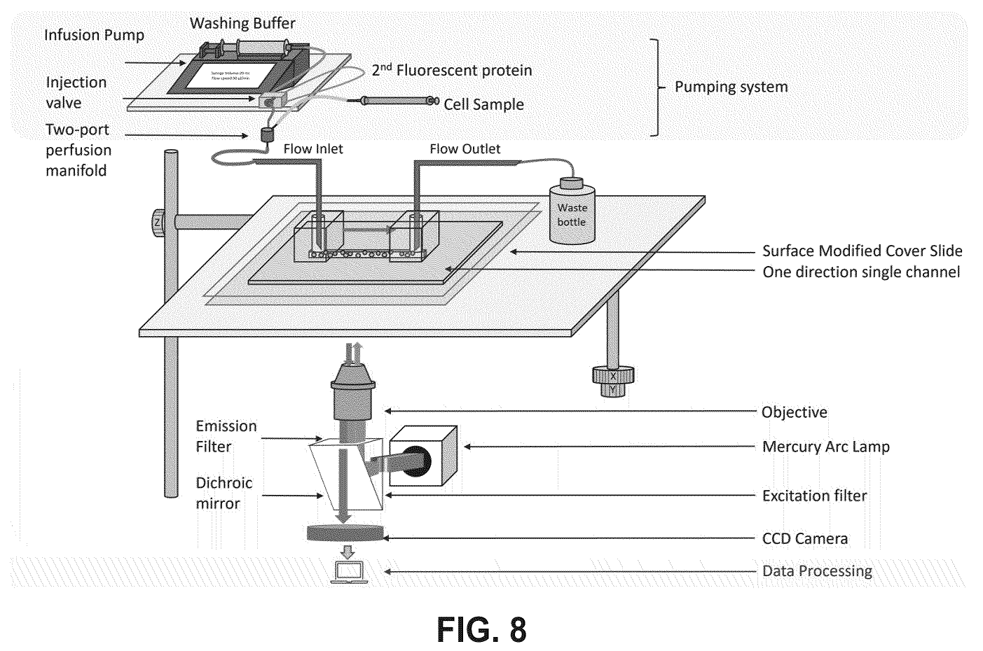

As shown in FIG. 8, the sample delivery and detection system consists of four parts: 1) Flow control: a syringe pump (Chemyx Fusion 100, Catalog #: 07100) provide constant flow rate (10 μL/mins); a 6-port injection valve (VICI Valco Instruments Co. Inc., #08N-0232H) for switching between washing buffer and the fluorescent protein; A 20 mL Sterile syringe for loading washing buffer and a 1 mL sterile syringe for loading cells samples (VWR #53548-008 & 53548-001). 2) Sensor chip: a homemade fluidic chip with one straight channel for sample testing and observing; 3) Image acquisition: an inverted microscope (Olympus IX81, 40×/0.75 NA objective) with a CMOS camera for recording bright field and fluorescence image sequences. 4) Data processing: MATLAB (R2022a) and Fiji Image J are used for image processing and data visualization.

### Microfluidic Chip Fabrication

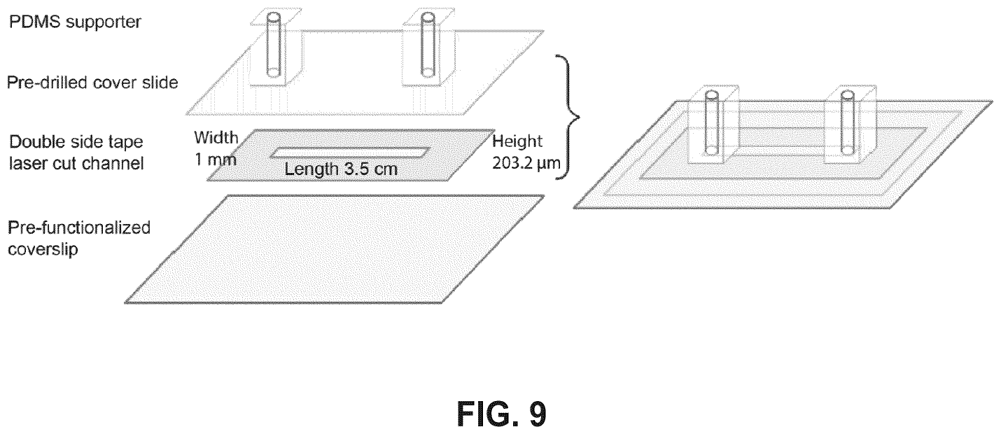

The microfluidic chip we used for the experiments included 4 components (FIG. 9). From the bottom to the top, there is a 25×50 mm coverslip (Mercedes Medical Glass Coverslips, Fisher Scientific, #15-184-03), a microfluidic channel defined by two layers of double-sided tapes (4 Mil/101.6 μm thick for each layer; #3M 444), top cover glass (Cole-Parmer, 22×40 mm, #UX-48511-10) with two 1 mm diameter pre-drilled holes as the inlet and outlet, and two polydimethylsiloxane (PDMS, Momentive Specialty Chemicals; #RTV-615-044) channel connector holders attached to the top cover slides via oxygen plasma treatment. The microfluidic channel defined by the double-sided tapes were designed in Adobe illustrator (2020) and fabricated with a laser cutter (Universal Laser System, VLS 4.15). The channel is linear, with 202.6 μm in height, 1000 μm in width, and 3.5 cm in length, and the total volume is 7.1 μL. PDMS channel connector holders were fabricated with classic protocol⁵⁸⁻⁶⁰. Briefly, 30 mL of the semi-solid polydimethylsiloxane was poured into a clean plastic petri dish with a flat surface, degassed in a vacuumed desiccator (for 30 mins), and baked in an oven at 60° C. for 60 minutes. Next, the hardened PDMS was peeled off from the dish, cut it into small cubes. A thorough hole is punched through each PDMS cube with a hole puncher (Robbins #RBP-075) for holding the stainless-steel flow channel connectors. The chip was assembled bottom up starting with a functionalized bottom glass, and followed by the double-sided tape defined channel, the top cover slide with holes were aligned with the channel end, and finally two PDMS holders were aligned with the top glass holes.

### Surface Chemistry

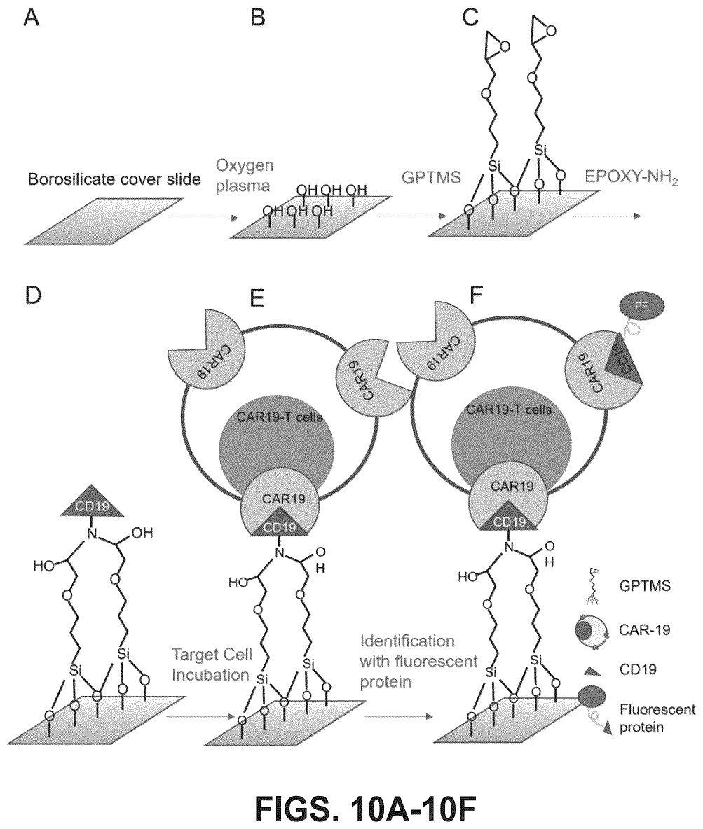

The process of surface modification of the sensor chip is depicted in FIG. 10A-10F. To capture the Jurkat CAR-19⁺ T cells, which express CD19 receptors on their membrane, CD19 protein was immobilized onto the sensing surface (i.e., top surface of the bottom glass). The conjugation of protein to the glass surface was achieved using either (3-glycidyloxypropyl) trimethoxylsilane (GPTMS) or 3-(2,3-Epoxypropoxy) propyltrimethoxysilane (GLYMO) (Sigma-Aldrich #440167) as a coupling agent⁶¹⁻⁶⁴. The oxirane ring in the coupling agent undergoes nucleophilic addition reaction with the amino group in the target protein, leading to covalent binding of the protein to the glass surface. The coverslips (22×50 mm) were cleaned sequentially with 99% isopropanol (VWR #: 67-63-0), deionized water (15 MΩ/cm, 0.22 μm filtration), and air-dried using a nitrogen stream. The coverslips were further cleaned using oxygen plasma cleaning (Harrick Plasma, #PDC001051102 High frequency, 2.96 W, 3 mins). The coupling agent, 1% (v/v) (3-glycidyloxypropyl) trimethoxylsilane (GPTMS) in anhydrous isopropanol (IPA), was freshly prepared one day before starting the surface modification. The plasma-treated glass coverslips were soaked in this solution overnight (24 hours) at room temperature in a glass staining jar, followed by cleaning with anhydrous IPA and distilled water, and drying using a nitrogen stream. The top coverslip and PDMS connector holders were also cleaned using the same washing steps. After the sensor chip was assembled, 0.1 mg/mL Human recombinant CD19 protein solution (Sino Biological; #11880-H08H), diluted with 50 mM sodium carbonate buffer (pH=9.6), was injected into the channel through the inlet and incubated for 30 minutes. The chip was gently washed with 1 mL 1× living cell imaging solution (Thermo Fisher Scientific #A14291DJ) and incubated with 1× Blocking Reagent (10%, v/v, Roche #11096176001) statically for 30 minutes at room temperature to block nonspecific binding sites. The chip was ready for use in the next 6 hours. All experiments were performed using freshly prepared slides and samples.

### Sample Preparation

To determine the density of blood cells, fresh whole blood samples were diluted 1000-fold with living cell imaging buffer to ensure that the maximum reading threshold of the cell counter was not exceeded. To prepare the Jurkat T cell samples, 2 mL of both wild-type (WT) Jurkat T cells and Jurkat CAR-19⁺ T cells were centrifuged at 300 g for 5 minutes. The supernatant was discarded, and the cells were re-diluted with living cell imaging buffer to eliminate potential interference from FBS and other culture medium components. The concentration of all cell samples was adjusted to ˜2×10³ cells/μL as a standard cell sample before any experiment. This standard sample was then diluted with living cell imaging buffer or human whole blood to reach final concentrations of 10³, 10², 10¹, and 10⁰ Jurkat CAR-19⁺ T cells/μL with a fixed 10 μL spike volume (10 μL/990 μL). There were three groups of spiking solutions: 1) Living cell imaging buffer. 2) Reagent red blood cells purchased from immucor (Catalog #: IG2338). Healthy human whole blood collected from healthy volunteers using K₂ EDTA tubes (BD, #23-021-015) (approved by IRB STUDY00008255). The whole-blood spiked samples were designed to mimic clinical patient samples.

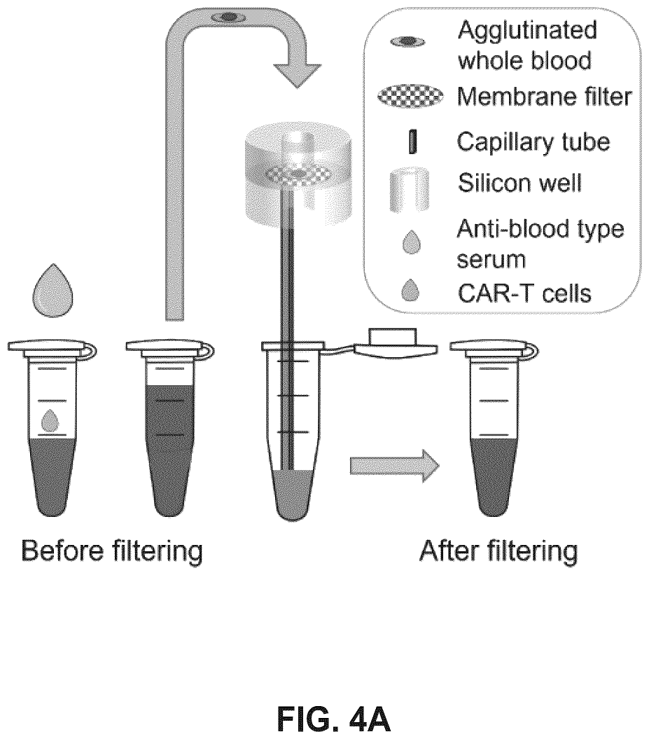

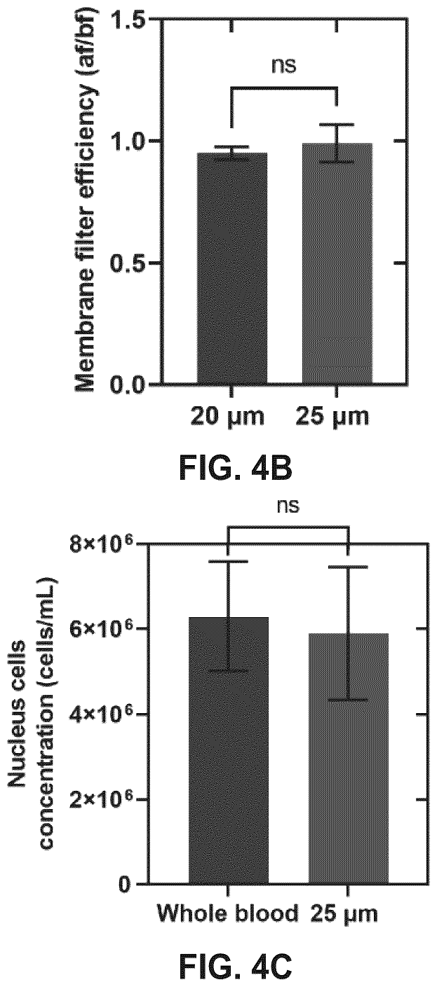

Next, 25 μL of anti-blood type solution (Ortho Clinical Diagnostic Inc; #6901934) was gently mixed with 50 μL of whole blood or reagent red blood cells spiked samples at room temperature for 30 mins. To remove any aggregated red blood cells, the spiked samples were transferred to a simple filter system (FIG. 4A). A hydrophilic membrane filter (Sterlitech #PCT25013100) with a 25 μm pore size was inserted between two reusable silicone sample wells (FlexiPERM micro12, #94.6011.436). A capillary tube (VitroCom #8270) located below the filter provided capillary force to extract the liquid portion containing non-agglutinated cells of the agglutinated sample mixture for measurement.

### Sample Loading and Processing

Following the preparation of the samples, the filtered sample was introduced into the sensor chip. The CD19 protein immobilized on the chip surface selectively captured the target CAR-19⁺ T cells. After 15 minutes of incubation, a washing buffer (living cell imaging solution) was perfused through the microfluidic system at a flow rate of 10 μL/min to remove the non-target cells from the region of interest. Two sets of images were acquired for each experiment, one prior to the washing step and the other after the washing step. In total, each set consisted of 30 images covering the entire length of the linear channel.

### Optical Image Acquisition and Processing

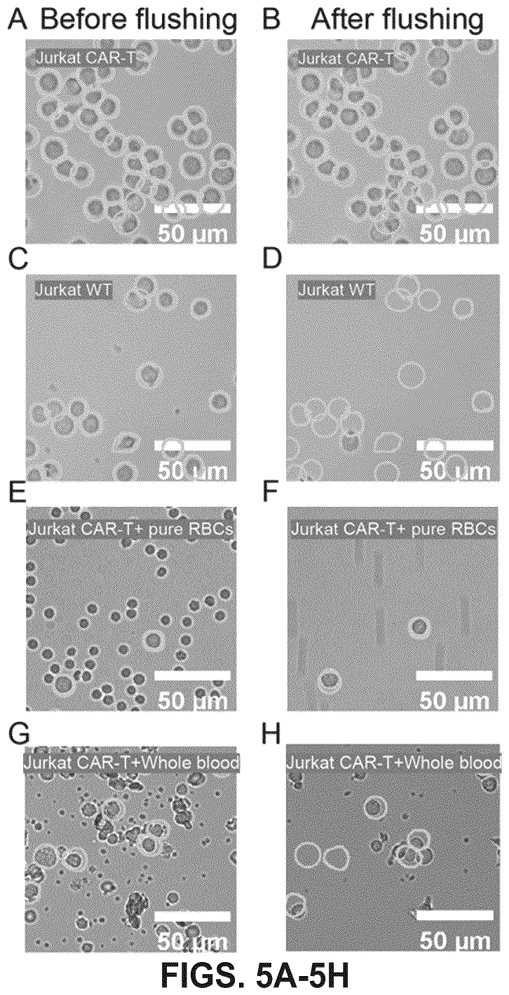

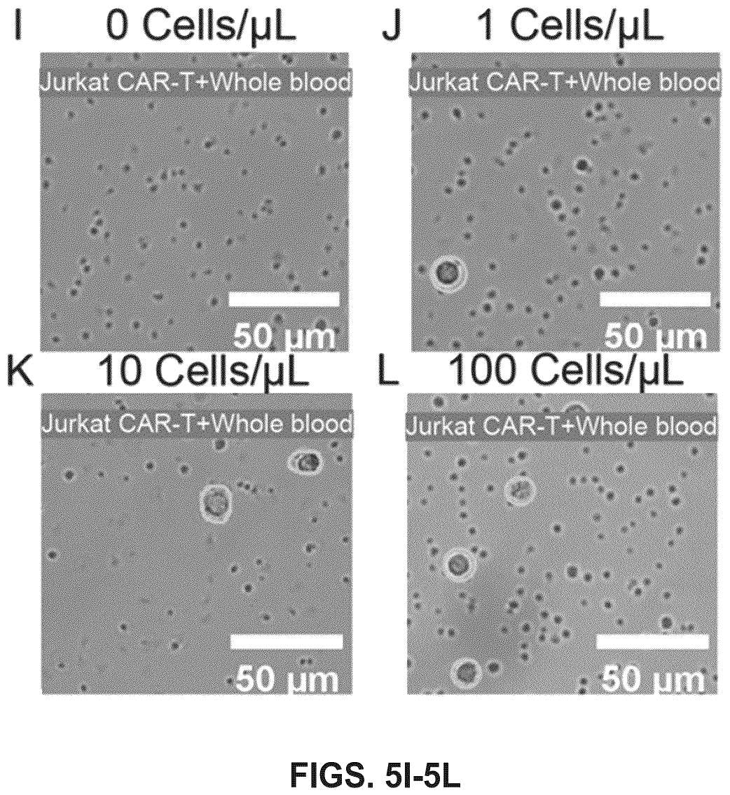

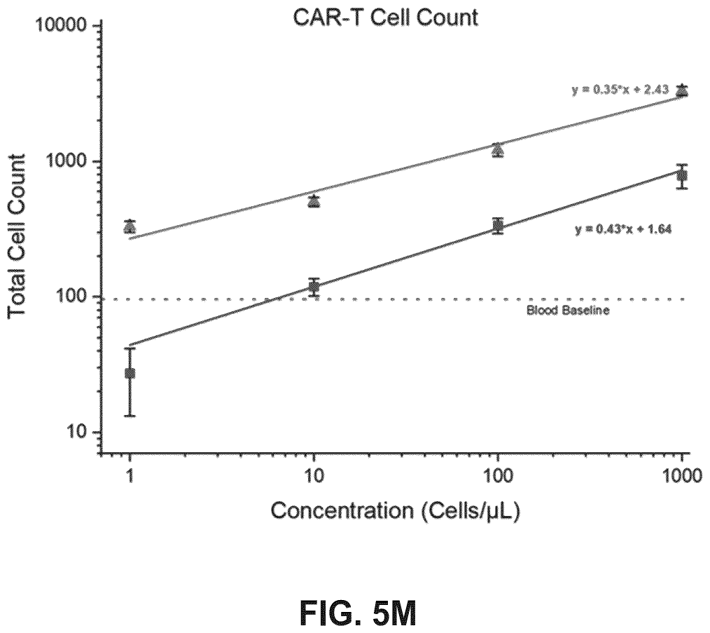

In this example, we used an inverted microscope (Olympus IX81, 40×/0.75 NA objective) in combination with a high-resolution CMOS camera (Hamamatsu ORCA-Flash 4.0 V3:C13440-20CU; Pixel size: 6.5 μm; View size: 2048 pixels×2048 pixels) to obtain bright and fluorescent images. A mercury light (Olympus U-LH100HG) was used as the fluorescence light source. An appropriate filter set (Excitation wavelength: 543±10, Emission wavelength: 593±40) was applied for fluorescence imaging. The bright field images were recorded with a 100 ms exposure time, while exposure time for fluorescence image is 4 s. To analyze the images, we utilized image-processing software (Fuji) to subtract background noise from the first (before flushing) and last (after flushing) images, identify the positions of the target cells in the first image, quantify the remaining cell fraction in the two images, and compare the results. (FIG. 5A-5H).

### Cell Culture

This example used two distinct cell lines, namely Jurkat CAR-19⁺ T cells and corresponding wild-type (WT) Jurkat T cells. Jurkat CAR-19⁺ T cells were engineered to express the anti-human CD19 gene, containing a single-chain variable fragment (scFv) derived from the FMC63 monoclonal antibody, which mimics the function of human CAR-19+ T cells. On the other hand, the WT cells do not express the CD19 antigen. Both cell lines were cultured and prepared in accordance with a previously published protocol. Specifically, the cells were cultured in Nunc EasYFlask 75 cm² (Thermo Fisher Scientific, Catalog #: 156472) using RPMI 1640 medium (Gibco, A1049101), supplemented with 10% (v/v) fetal bovine serum (FBS) (Gibco, A31606-02) and 1% (v/v) penicillin-streptomycin (Corning, 30-002-CI). Cell density was maintained at 1×10⁶ cells/mL monitoring with an automated cell counter (Logos Biosystems, LUNA-II™, L40001).

### Immunostaining

To identify the cells retained in a microfluidic chip, Human CD19-PE conjugated protein was injected into the chip system right after the flushing process and incubated for 30 minutes. Then the injection valve was switched to the washing buffer to wash away the excess fluorescent dye for 60 seconds, and the chip was ready for imaging.

### Fluorescence-Activated Cell Sorting (FACS)

The Fluorescence-activated cell sorting (FACS) process was conducted according to a standard protocol. In brief, we centrifuged 10⁶ cells at 300 g for 5 mins and washed them three times with living cell imaging solutions to ensure accurate cell counts using the Logos LUNA-II cell counter. The buffer-diluted Jurkat WT/CAR 19-T cells were then incubated with 1000 μL 0.5% BSA (Sigma Aldrich #05470) as the blocking buffer for 30 mins, followed by three centrifuging and washing steps. Subsequently, the cells were incubated with 100 μL Human CD19-PE conjugated protein (1:50) for another 30 minutes. The cells were then subjected to three centrifuging and washing steps to eliminate excessive fluorescent protein influence. The cells were then analyzed in the Attune NXT Flow Cytometer.

### Machine Learning Algorithm

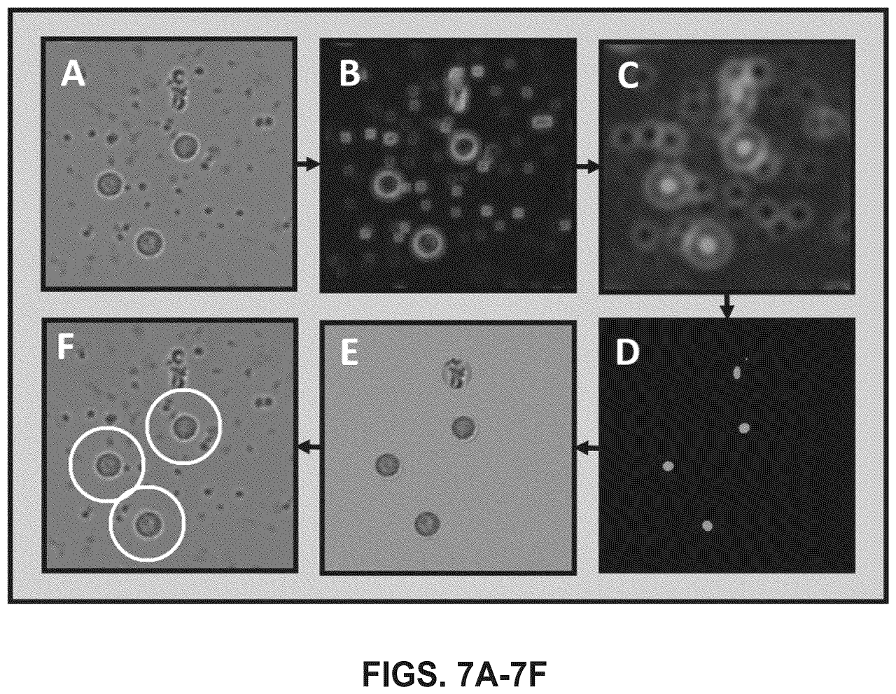

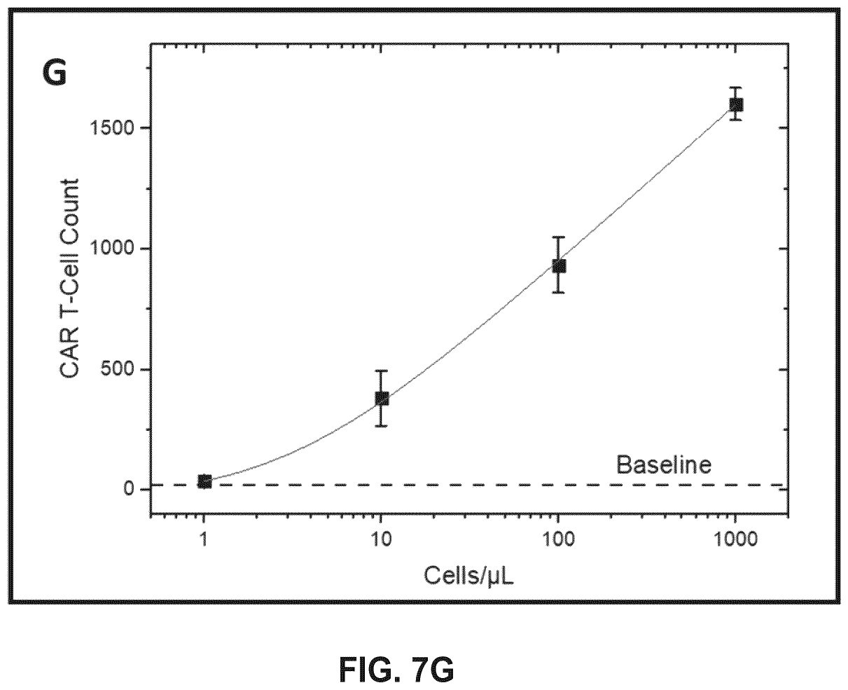

In this example, we employed the AlexNet model, a widely tested convolutional neural network that has been successfully applied to image classification tasks, to differentiate CAR-T cells from normal T cells based on fluorescence signals. To accomplish this, we adapted the algorithm from a pre-trained AlexNet model using MATLAB. This approach was chosen for its ability to save time and maintain model accuracy. To create a training dataset, we labeled images of positive and negative cells using the Phycoerythrin (PE)-labeled recombinant CD19 protein. The dataset was then manually curated by experienced researchers based on the following criteria: a) the cell was deemed alive and positive based on bright field images of an intact membrane, and b) CAR-T cells exhibited bright fluorescent spots, primarily concentrated at the cell membrane's edge (as shown in FIG. 7B). The resulting trained model was able to accurately differentiate between positive and negative cells, as illustrated in FIG. 7.

### Statistics

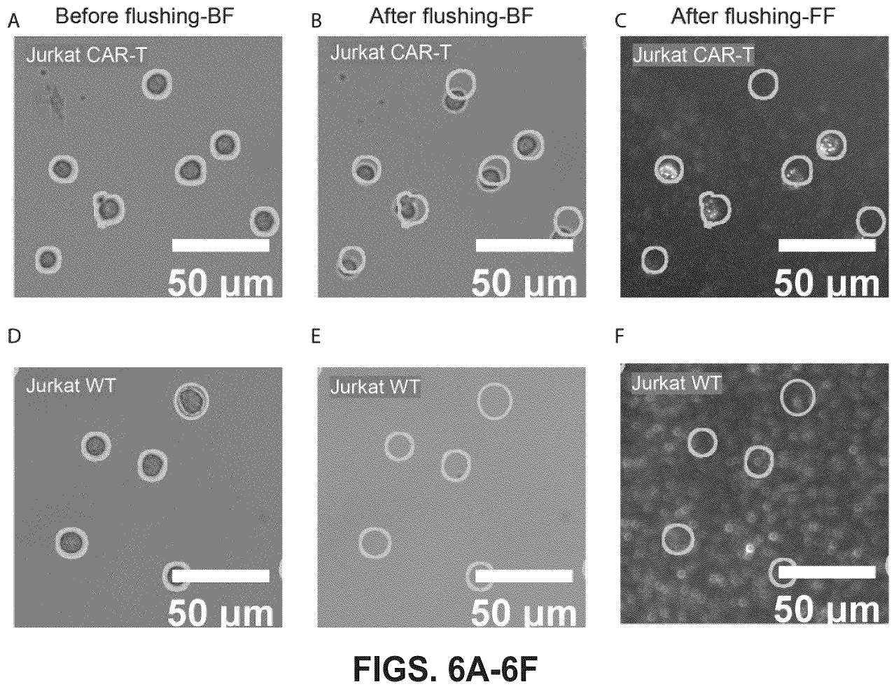

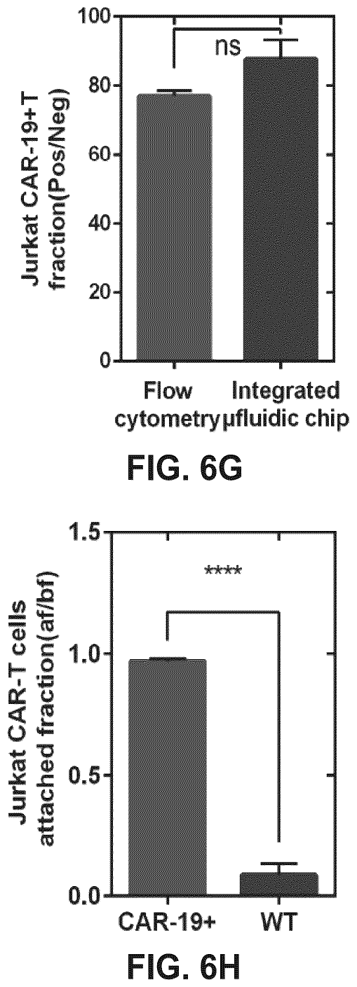

The number of cells retained on the ROI surface after flushing were considered as the Positive cells in our system, which were also collected as the Jurkat CAR-T cells attached number in our calibration curve (FIG. 5M). The percentage of positive cells (FIG. 6H, left bar) was determined by dividing the number of retaining cells after flushing by the number before flushing. All data are presented as mean±standard deviation (SD).

Statistical analyses were performed using a two-tailed Student's t-test for comparisons between two groups and one-way ANOVA for comparisons among multiple groups, such as the calibration curve. All experiments were conducted at least three times, and representative experiments were shown. Differences were considered significant at P<0.05. Data analysis was performed using GraphPad Prism Version 9.2.0.

### Results

### Centrifuge-Free, Agglutination-Based RBCs Removal

The ratio between RBCs and WBCs in whole blood is typically around 600:1. Therefore, it is necessary to remove RBCs from whole blood samples for evaluating peripheral blood mononuclear cells (PBMCs), platelets, or molecules in plasma. Although the centrifugation-based separation method is accurate and efficient, it requires a bulky high-speed centrifuge that is not compatible with point-of-care setting. As an alternative method that does not require centrifugation, ACK (Ammonium-Chloride-Potassium) lysis buffer has been widely accepted for lysing red blood cells in biological samples. However, the method is not ideal, as the lysis buffer also lyses a portion of WBCs, resulting in reduced recovery rate.

Here, we introduced the agglutination assay for centrifuge-free RBCs' removal from whole blood sample. RBC membrane protein, which carries over 2×10⁶ A, B, and H antigens per RBC, plays a critical role in the process of normal human blood agglutination. The specific anti-blood type antibodies exist in the corresponding blood serum. We use the antibody triggered RBC agglutination coupled with a large pore size filter as a simply way to remove the aggregated RBCs from the rest of the blood components including WBCs. For proof of principle test, we mixed 50 μL whole blood sample with 25 μL corresponding anti-blood type serum for 30 minutes. Then we transferred the agglutinated sample into a simple filtration system (FIG. 4A) with a polycarbonate (PCTE) membrane filter placed in the center of a well to block the agglutinated RBCs. The rest of the blood will be extracted through a capillary tube and collected in a test tube.

To determine the proper pore size of the membrane filter, we tested two filters with different pore size (20 μm and 25 μm) to evaluate the filtration efficiency of buffer-diluted Jurkat CAR-T cells and Jurkat WT cells. The concentration of white blood cells (WBCs) was measured before and after filtration (FIG. 4B). The results showed that both membrane filters achieved over 95% filtration efficiency, with no significant difference (P=0.2304). Based on the average size of lymphocytes, which ranges from 7 to 15 μm⁷⁵, we selected the 25 μm PCTE membrane filter for subsequent experiments to minimize the loss of target cells.

However, it's more challenging to evaluate the membrane filter efficiency for samples spiked with whole blood due to high cell density, which exceeds the LUNA II automatic cell counter's testing threshold. It's difficult to make an accurate distinguishment between RBCs and the other contents without labeling or only by dimension differences. Therefore, we used the gold standard hemocytometer counting method to evaluate the filtration efficiency. To facilitate the recognition, 0.1% Triton X-100 was used as the permeabilizer, and 0.4% Trypan Blue was applied as the DNA indicator to stain the nuclei. The results of the cell count before and after filtration showed no significant difference between the original undiluted whole blood samples and the filtered samples after agglutination (P=0.6353), indicating a high recovery rate using this filtration system (FIG. 4C)

### CAR-T Cell Detection Principle, Workflow, and Results

A detailed description of the methodology can be found in the Methods section. Following a 15-minute incubation period, a washing buffer, specifically a living cell imaging solution, was introduced into the flow-driven system at a flow rate of 10 μL/min to detach non-target cells from the region of interest. Two images were captured for each experiment, before and after the washing step, as depicted in FIGS. 5A to 5H. Quantifiable numbers of cells bound to the CAR-19⁺T cell capture zone were observed in buffer-diluted pure Jurkat CAR-19+ T cells and blood samples spiked with Jurkat CAR-19+ T cells. However, in samples containing wild-type Jurkat T cells, over 90% of previously attached cells were removed after flushing, indicating the rest of these attached cells were non-specific binding to the sensor surface.

To illustrate the utility of our optical imaging-based CAR-T detection platform, we spiked various concentrations of Jurkat CAR-19⁺ T cells into undiluted whole blood obtained from healthy human donors. The number of cells attached to the modified surface in the entire channel (30 regions of interest) was counted after the washing step. Four different concentrations of Jurkat CAR-19⁺ T cells spiked in whole blood after flushing are shown in FIGS. 5I to 5L. Two standard curves (FIG. 5M) were generated by measuring a range of Jurkat CAR-19+ T cell concentrations in whole blood spiked and buffer-diluted samples. A linear relationship was observed between the concentrations of Jurkat-CAR19+ T cells in the sample and the number of captured cells (R_{wb}²=0.9852, R_{buffer}²=0.9602).

The average cell attachment levels in whole blood spiked samples were found to be significantly lower with a larger error bar than buffer diluted samples, which could be attributed to the presence of abundant proteins such as human albumin, that block the binding sites and prevent the attachment of target cells. Additionally, platelet interference cannot be ignored as they play a crucial role in the process of blood clotting and can directly interact with WBCs through surface molecules and soluble mediators. This interaction promotes leukocyte recruitment and activation, as well as platelet aggregation and activation. Our study found that non-fresh samples, especially those preserved under 4° C. overnight, exhibited strong non-specific binding and an increasing number of attached platelets, introducing high non-specific binding. This could be due to the stimulation of platelets by cold or low oxygen levels, which results in increased platelet activity. While the results related to whole blood samples exhibit a linear progression trend, it was challenging to conduct experiments with whole blood samples due to the complex and delicate nature of the procedure, including addressing the issues of non-specific binding and platelet interference.

### Validation of Cell Identity

The precision and accuracy of CAR T-cell detection on a chip can be affected by two factors: 1) the heterogenous expression level of transfected cells, which may lead to the absence of CARs in captured Jurkat CAR-T cells; 2) non-specific binding of other white blood cells in the sample. While image analysis algorithms can distinguish these negative cells based on their morphology (granularity, vesicles, membrane, clusters), experimental validation is required to confirm the identity of all captured cells. To address this issue, we used fluorescent labeling to identify Jurkat CAR T-cells captured on the sensor chip and validated the results using flow cytometry, which is considered the gold standard methodology (FIG. 6). We captured cultured Jurkat and Jurkat CAR-19⁺ T-cells on the chip and stained them with a fluorescent-labeled CD19-PE recombinant protein. We then captured and analyzed brightfield and fluorescence images (FIG. 6A-6F). The results indicated that 96.96% of Jurkat CART-19+T cells were retained on the surface, with a strong fluorescence signal in 87.67% of positive cells (FIG. 6G). In contrast, a small percentage (8.89%) of Jurkat T cells remained bound to the surface, but did not emit fluorescence, suggesting the absence of CARs. The percentage of CAR-19+ T-cells in each Jurkat T cell sample was also validated by flow cytometry. Our sensor exhibited a slightly higher percentage of captured Jurkat CAR-19+ T-cells, likely due to the CD19-functionalized surface that favors the capture of Jurkat CAR-19+ T-cells and improves detection specificity. These results confirm the specificity of the capture surface in our sensor.

Although fluorescent-activated cell sorting (FACS) or fluorescent staining is considered the gold standard for validating cell identity, the interpretation of results can be challenging due to background fluorescence caused by the presence of red blood cells, platelets, and other cellular components in whole blood samples. Additionally, the fluorescent protein/dye bleaching effects can also contribute to background noise and reduce the signal-to-noise ratio. The amount of staining protein/antibody used for FACS can also affect the precision of results, and dead or broken cells can emit self-fluorescence. Moreover, the expression level of chimeric antigen receptors can vary significantly, further adding to the complexity of obtaining accurate results. Therefore, overcoming these challenges is critical for accurate identification and quantification of cells in whole blood samples using FACS or fluorescent staining techniques. amounts of fluorescent protein bond with a non-fully blocked surface created background noises. Therefore, to create a more accurate and precise bright field imaging-based CAR T-cell detection, we tried and developed machine learning algorithms to identify CAR 19⁺ T-cells from all captured cells based on optical-pixel-based morphological characteristics (size, internal granularity complexity, cellular membrane contour, among others)⁹⁵. We use fluorescently labeled Jurkat CAR-19⁺ T-cells for direct visualization and morphological classification with the AlexNet model to train our algorithm. AlexNet is a widely used and tested convolutional neural network and performs well in classifying 1,000 different classes in ImageNet. To distinguish CAR T-cells from untransduced T cells, we applied the transfer learning to a pre-trained AlexNet model in MATLAB using our cell data (FIG. 7). The first set of validated images was verified manually by experienced researchers, giving an accuracy of 95% in distinguishing positive and negative cells. The model was further validated using pure positive (Jurkat-CAR-19⁺ T cells) and negative samples (wild-type Jurkat cells). The classification results were consistent with that of the flow cytometry experiment. We obtained less than a 10% error compared with the flow cytometry result, indicating the correct classification of our model.

### Conclusion

Our solution provided a potential POC testing platform for monitoring CAR-T immunotherapy-treated patients' CAR-T cells number by tracking the real-time dynamic cell concentration change bound to the modified surface with only 50 μL undiluted whole blood sample. The total material cost of fabricating the fluidic chip was less than $9. The total assay time from sample loading to automatic readout is less than 1 hour. We can achieve a low detection limit of 1 cell/μL, which also indicates a promising broaden application of counting circulating rare tumor cells (CTCs) at an early developing stage. The cytokine release syndrome, which reacts as a double-side blade, indicates CAR-T therapy's efficacy and efficiency, can be triggered by the expansion of CAR-T cells in the patients' bodies. In further development, cytokines detections via our previously developed optical imaging-based digital immunoassay can be integrated with CAR-T cells counting with a multiple zone fluidic chip.

Some further aspects are also defined in the following clauses:

Clause 1: A method of differentiating cell types in a cell population, the method comprising: removing at least some non-Chimeric Antigen Receptor (CAR)-T cells from a fluidic sample obtained from a subject without centrifuging the fluidic sample to produce a purified fluidic sample, wherein the fluidic sample comprises CAR-T cells and the non-CAR-T cells; capturing cells in the purified fluidic sample on a surface that comprises one or more binding moieties that bind at least to the CAR-T cells to produce a captured cell population; and, distinguishing the CAR-T cells from the non-CAR-T cells in the captured cell population using a trained machine learning model to produce a captured CAR-T cell population data set, thereby differentiating the cell types in the cell population.

Clause 2: The method of Clause 1, wherein the binding moieties are conjugated to the surface.

Clause 3: The method of Clause 1 or Clause 2, wherein the binding moieties bind to receptors on the CAR-T cells.

Clause 4: The method of any of Clauses 1-3, wherein the binding moieties comprise one or more anti-CAR-T cell antibodies or antigen binding portions thereof.

Clause 5: The method of any of Clauses 1-4, wherein the binding moieties comprise one or more antigens or functional portions thereof.

Clause 6: The method of any of Clauses 1-5, wherein the antigens comprise recombinant antigens.

Clause 7: The method of any of Clauses 1-6, wherein the antigens comprise CD19 molecules and wherein the CAR-T cells comprise CD19-targeted CAR-T cells.

Clause 8: The method of any of Clauses 1-7, comprising counting the CAR-T cells in the captured cell population.

Clause 9: The method of any of Clauses 1-8, wherein the trained machine learning model is configured to distinguish the CAR-T cells from the non-CAR-T cells in the captured cell population based at least in part on one or more morphological characteristics of the CAR-T cells and/or the non-CAR-T cells.

Clause 10: The method of any of Clauses 1-9, wherein the distinguishing step further comprises taking one or more images of the captured cell population bound to the binding moieties using an optical imaging mechanism to produce a captured cell population image data set when the fluidic sample flows through the cavity via the opening, and wherein the trained machine learning model uses the captured cell population image data set to produce the captured CAR-T cell population data set.

Clause 11: The method of any of Clauses 1-10, wherein a cavity of a microfluidic device comprises the surface.

Clause 12: The method of any of Clauses 1-11, further comprising quantifying the CAR-T cells in the captured CAR-T cell population data set to produce a quantified CAR-T cell data set.

Clause 13: The method of any of Clauses 1-12, comprising administering, or altering an administration of, at least one therapy to the subject based at least in part on the quantified CAR-T cell data set.

Clause 14: The method of any of Clauses 1-13, further comprising detecting one or more cytokines present in the fluidic sample.

Clause 15: The method of any of Clauses 1-14, comprising detecting the cytokines present in the fluidic sample using gold nanoparticle labeled detection antibodies or antigen binding portions thereof.

Clause 16: The method of any of Clauses 1-15, further comprising quantifying the cytokines present in the fluidic sample to produce a quantified cytokine data set.

Clause 17: The method of any of Clauses 1-16, comprising administering, or altering an administration of, at least one therapy to the subject based at least in part on the quantified cytokine data set.

Clause 18: The method of any of Clauses 1-17, wherein the non-CAR-T cells comprise red blood cells (RBCs).

Clause 19: The method of any of Clauses 1-18, wherein the at least some non-CAR-T cells are present in the fluidic sample in a form of agglutinated cells and wherein the removing step comprises filtering the agglutinated cells from the fluidic sample using a filtering mechanism.

Clause 20: The method of any of Clauses 1-19, wherein the filtering mechanism comprises a filter having a pore size of no more than about 10 μm.

Clause 21: The method of any of Clauses 1-20, wherein the fluidic sample has a volume of between about 25 μL and about 75 μL.

Clause 22: The method of any of Clauses 1-21, wherein the fluidic sample is a whole blood sample.

Clause 23: The method of any of Clauses 1-22, comprising agglutinating red blood cells (RBCs) in the whole blood sample prior to and/or concurrent with removing the at least some CAR-T cells from the fluidic sample.

Clause 24: The method of any of Clauses 1-23, comprising agglutinating the RBCs using at least one anti-blood type antibody.

Clause 25: A device, comprising: a housing structure that comprises a body structure comprising at least one cavity at least partially disposed within the body structure and an opening that fluidly communicates with the cavity, wherein at least one surface of the cavity comprises one or more binding moieties that bind at least to Chimeric Antigen Receptor (CAR)-T cells; a filter mechanism operably connected, or connectable, to the housing structure, which filter mechanism is configured to prevent at least some non-CAR-T cells in a fluidic sample from contacting the surface of the cavity when the fluidic sample flows through the cavity via the opening; a detector operably connected, or connectable, to the housing structure, which detector is configured to detect a captured cell population bound to the binding moieties when the fluidic sample flows through the cavity via the opening; and, a controller operably connected, or connectable, to the housing structure, which controller comprises, or is configured to communicate with, a trained machine learning model that distinguishes the CAR-T cells from the non-CAR-T cells in the captured cell population to produce a captured CAR-T cell population data set when the fluidic sample flows through the cavity via the opening.

Clause 26: The device of Clause 25, wherein the filtering mechanism comprises a filter having a pore size of no more than about 10 μm.

Clause 27: The device of Clause 25 or Clause 26, wherein the binding moieties are conjugated to the surface.

Clause 28: The device of any of Clauses 25-27, wherein the binding moieties bind to receptors on the CAR-T cells.

Clause 29: The device of any of Clauses 25-28, wherein the binding moieties comprise one or more anti-CAR-T cell antibodies or antigen binding portions thereof.

Clause 30: The device of any of Clauses 25-29, wherein the binding moieties comprise one or more antigens or functional portions thereof.

Clause 31: The device of any of Clauses 25-30, wherein the antigens comprise recombinant antigens.

Clause 32: The device of any of Clauses 25-31, wherein the antigens comprise CD19 molecules and wherein the CAR-T cells comprise CD19-targeted CAR-T cells.

Clause 33: The device of any of Clauses 25-32, wherein a cartridge comprises the body structure and wherein the housing structure is configured to reversibly receive the cartridge.

Clause 34: The device of any of Clauses 25-33, wherein the detector comprises an optical imaging mechanism that is configured to take one or more images of the captured cell population bound to the binding moieties to produce a captured cell population image data set when the fluidic sample flows through the cavity via the opening, and wherein the trained machine learning model is configured to use the captured cell population image data set to produce the captured CAR-T cell population data set.

Clause 35: The device of any of Clauses 25-34, wherein the trained machine learning model is configured to distinguish the CAR-T cells from the non-CAR-T cells in the captured cell population based at least in part on one or more morphological characteristics of the CAR-T cells and/or the non-CAR-T cells.

Clause 36: The device of any of Clauses 25-35, wherein the controller is configured to transmit at least a portion of the captured CAR-T cell population data set to a remote device or system.

Clause 37: The device of any of Clauses 25-36, wherein the controller further comprises, or is configured to further communicate with, one or more non-transient instructions, which when executed by a processor, further perform at least: counting the CAR-T cells in the captured cell population.

Clause 38: The device of any of Clauses 25-37, wherein the controller further comprises, or is configured to further communicate with, one or more non-transient instructions, which when executed by a processor, further perform at least: quantifying the CAR-T cells in the captured CAR-T cell population data set to produce a quantified CAR-T cell data set.

Clause 39: The device of any of Clauses 25-38, wherein the controller further comprises, or is configured to further communicate with, one or more non-transient instructions, which when executed by a processor, further perform at least: outputting at least one therapy recommendation based at least in part on the quantified CAR-T cell data set.

Clause 40: The device of any of Clauses 25-39, wherein the detector is further configured to detect one or more cytokines present in the fluidic sample when the fluidic sample flows through the cavity via the opening.

Clause 41: The device of any of Clauses 25-40, wherein the controller further comprises, or is configured to further communicate with, one or more non-transient instructions, which when executed by a processor, further perform at least: quantifying the cytokines present in the fluidic sample to produce a quantified cytokine data set.

Clause 42: The device of any of Clauses 25-41, wherein the controller further comprises, or is configured to further communicate with, one or more non-transient instructions, which when executed by a processor, further perform at least: outputting at least one therapy recommendation based at least in part on the quantified cytokine data set.

Clause 43: The device of any of Clauses 25-42, wherein the filter mechanism is configured to prevent at least some of the non-CAR-T cells present in the fluidic sample in a form of agglutinated cells from contacting the surface of the cavity when the fluidic sample flows through the cavity via the opening.

Clause 44: The device of any of Clauses 25-43, comprising a fluid conveyance mechanism operably connected, or connectable, to the housing structure and/or to the cartridge, which fluid conveyance mechanism is configured to flow the fluidic sample through the cavity via the opening.

Clause 45: The device of any of Clauses 25-44, wherein the fluidic sample has volume of between about 25 μL and about 75 μL when the fluidic sample flows through the cavity via the opening.

Clause 46: The device of any of Clauses 25-45, wherein the controller is configured to wirelessly communicate with a computer that comprises the trained machine learning model

Clause 47: The device of any of Clauses 25-46, wherein the device is hand-held.

Clause 48: The device of any of Clauses 25-47, wherein the device comprises a point-of-care device.

Clause 49: The cartridge of any of Clauses 25-48.

Clause 50: A kit comprising the cartridge of Clauses 25-49.

Clause 51: A kit comprising the device of any of Clauses 25-50.

Clause 52: A system, comprising: a fluidic sample receiving area, wherein at least one surface of the fluidic sample receiving area comprises one or more one or more binding moieties that bind at least to receptors on Chimeric Antigen Receptor (CAR)-T cells; a filter mechanism operably connected, or connectable, to the fluidic sample receiving area and/or to another system component, which filter mechanism is configured to prevent at least some non-CAR-T cells in a fluidic sample from contacting the surface of the fluidic sample receiving area when the fluidic sample flows through the fluidic sample receiving area; a detector operably connected, or connectable, to the fluidic sample receiving area and/or to another system component, which detector is configured to detect a captured cell population bound to the binding moieties when the fluidic sample flows through the fluidic sample receiving area; and, a controller operably connected, or connectable, to the fluidic sample receiving area and/or to another system component, which controller comprises, or is configured to communicate with, a trained machine learning model that distinguishes the CAR-T cells from the non-CAR-T cells in the captured cell population when the fluidic sample flows through the fluidic sample receiving area.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of differentiating cell types in a cell population, the method comprising:
removing at least some non-Chimeric Antigen Receptor (CAR)-T cells from a fluidic sample obtained from a subject without centrifuging the fluidic sample to produce a purified fluidic sample, wherein the fluidic sample comprises CAR-T cells and the non-CAR-T cells;
capturing cells in the purified fluidic sample on a surface that comprises one or more binding moieties that bind at least to the CAR-T cells to produce a captured cell population; and,
distinguishing the CAR-T cells from the non-CAR-T cells in the captured cell population using a trained machine learning model to produce a captured CAR-T cell population data set, thereby differentiating the cell types in the cell population.

2. The method of claim 1, wherein the binding moieties are conjugated to the surface.

3. The method of claim 1, wherein the binding moieties bind to receptors on the CAR-T cells.

4. The method of claim 1, wherein the binding moieties comprise one or more anti-CAR-T cell antibodies or antigen binding portions thereof.

5. The method of claim 1, wherein the binding moieties comprise one or more antigens or functional portions thereof.

6. The method of claim 5, wherein the antigens comprise recombinant antigens.

7. The method of claim 5, wherein the antigens comprise CD19 molecules and wherein the CAR-T cells comprise CD19-targeted CAR-T cells.

8. The method of claim 1, comprising counting the CAR-T cells in the captured cell population.

9. The method of claim 1, wherein the trained machine learning model is configured to distinguish the CAR-T cells from the non-CAR-T cells in the captured cell population based at least in part on one or more morphological characteristics of the CAR-T cells and/or the non-CAR-T cells.

10. The method of claim 1, wherein the distinguishing step further comprises taking one or more images of the captured cell population bound to the binding moieties using an optical imaging mechanism to produce a captured cell population image data set when the fluidic sample flows through the cavity via the opening, and wherein the trained machine learning model uses the captured cell population image data set to produce the captured CAR-T cell population data set.

11. The method of claim 1, wherein a cavity of a microfluidic device comprises the surface.

12. The method of claim 1, further comprising quantifying the CAR-T cells in the captured CAR-T cell population data set to produce a quantified CAR-T cell data set.

13. The method of claim 12, comprising administering, or altering an administration of, at least one therapy to the subject based at least in part on the quantified CAR-T cell data set.

14. The method of claim 1, further comprising detecting one or more cytokines present in the fluidic sample.

15. The method of claim 14, comprising detecting the cytokines present in the fluidic sample using gold nanoparticle labeled detection antibodies or antigen binding portions thereof.

16. The method of claim 14, further comprising quantifying the cytokines present in the fluidic sample to produce a quantified cytokine data set.

17. The method of claim 16, comprising administering, or altering an administration of, at least one therapy to the subject based at least in part on the quantified cytokine data set.

18. The method of claim 1, wherein the non-CAR-T cells comprise red blood cells (RBCs).

19. The method of claim 1, wherein the at least some non-CAR-T cells are present in the fluidic sample in a form of agglutinated cells and wherein the removing step comprises filtering the agglutinated cells from the fluidic sample using a filtering mechanism.

20. The method of claim 19, wherein the filtering mechanism comprises a filter having a pore size of no more than about 10 μm.

21. The method of claim 1, wherein the fluidic sample has a volume of between about 25 μL and about 75 μL.

22. The method of claim 1, wherein the fluidic sample is a whole blood sample.

23. The method of claim 22, comprising agglutinating red blood cells (RBCs) in the whole blood sample prior to and/or concurrent with removing the at least some CAR-T cells from the fluidic sample.

24. The method of claim 23, comprising agglutinating the RBCs using at least one anti-blood type antibody.

25. A device, comprising:
a housing structure that comprises a body structure comprising at least one cavity at least partially disposed within the body structure and an opening that fluidly communicates with the cavity, wherein at least one surface of the cavity comprises one or more binding moieties that bind at least to Chimeric Antigen Receptor (CAR)-T cells;
a filter mechanism operably connected, or connectable, to the housing structure, which filter mechanism is configured to prevent at least some non-CAR-T cells in a fluidic sample from contacting the surface of the cavity when the fluidic sample flows through the cavity via the opening;
a detector operably connected, or connectable, to the housing structure, which detector is configured to detect a captured cell population bound to the binding moieties when the fluidic sample flows through the cavity via the opening; and,
a controller operably connected, or connectable, to the housing structure, which controller comprises, or is configured to communicate with, a trained machine learning model that distinguishes the CAR-T cells from the non-CAR-T cells in the captured cell population to produce a captured CAR-T cell population data set when the fluidic sample flows through the cavity via the opening.

26. The device of claim 25, wherein the filtering mechanism comprises a filter having a pore size of no more than about 10 μm.

27. The device of claim 25, wherein the binding moieties are conjugated to the surface.

28. The device of claim 25, wherein the binding moieties bind to receptors on the CAR-T cells.

29. The device of claim 25, wherein the binding moieties comprise one or more anti-CAR-T cell antibodies or antigen binding portions thereof.

30. The device of claim 25, wherein the binding moieties comprise one or more antigens or functional portions thereof.

31. The device of claim 30, wherein the antigens comprise recombinant antigens.

32. The device of claim 30, wherein the antigens comprise CD19 molecules and wherein the CAR-T cells comprise CD19-targeted CAR-T cells.

33. The device of claim 25, wherein a cartridge comprises the body structure and wherein the housing structure is configured to reversibly receive the cartridge.

34. The device of claim 25, wherein the detector comprises an optical imaging mechanism that is configured to take one or more images of the captured cell population bound to the binding moieties to produce a captured cell population image data set when the fluidic sample flows through the cavity via the opening, and wherein the trained machine learning model is configured to use the captured cell population image data set to produce the captured CAR-T cell population data set.

35. The device of claim 25, wherein the trained machine learning model is configured to distinguish the CAR-T cells from the non-CAR-T cells in the captured cell population based at least in part on one or more morphological characteristics of the CAR-T cells and/or the non-CAR-T cells.

36. The device of claim 25, wherein the controller is configured to transmit at least a portion of the captured CAR-T cell population data set to a remote device or system.

37. The device of claim 25, wherein the controller further comprises, or is configured to further communicate with, one or more non-transient instructions, which when executed by a processor, further perform at least:
counting the CAR-T cells in the captured cell population.

38. The device of claim 25, wherein the controller further comprises, or is configured to further communicate with, one or more non-transient instructions, which when executed by a processor, further perform at least:
quantifying the CAR-T cells in the captured CAR-T cell population data set to produce a quantified CAR-T cell data set.

39. The device of claim 38, wherein the controller further comprises, or is configured to further communicate with, one or more non-transient instructions, which when executed by a processor, further perform at least:
outputting at least one therapy recommendation based at least in part on the quantified CAR-T cell data set.

40. The device of claim 25, wherein the detector is further configured to detect one or more cytokines present in the fluidic sample when the fluidic sample flows through the cavity via the opening.

41. The device of claim 40, wherein the controller further comprises, or is configured to further communicate with, one or more non-transient instructions, which when executed by a processor, further perform at least:
quantifying the cytokines present in the fluidic sample to produce a quantified cytokine data set.

42. The device of claim 41, wherein the controller further comprises, or is configured to further communicate with, one or more non-transient instructions, which when executed by a processor, further perform at least:
outputting at least one therapy recommendation based at least in part on the quantified cytokine data set.

43. The device of claim 25, wherein the filter mechanism is configured to prevent at least some of the non-CAR-T cells present in the fluidic sample in a form of agglutinated cells from contacting the surface of the cavity when the fluidic sample flows through the cavity via the opening.

44. The device of claim 25, comprising a fluid conveyance mechanism operably connected, or connectable, to the housing structure and/or to the cartridge, which fluid conveyance mechanism is configured to flow the fluidic sample through the cavity via the opening.

45. The device of claim 25, wherein the fluidic sample has volume of between about 25 μL and about 75 μL when the fluidic sample flows through the cavity via the opening.

46. The device of claim 25, wherein the controller is configured to wirelessly communicate with a computer that comprises the trained machine learning model.

47. The device of claim 25, wherein the device is hand-held.

48. The device of claim 25, wherein the device comprises a point-of-care device.

49. The cartridge of claim 33.

50. A kit comprising the cartridge of claim 49.

51. A kit comprising the device of claim 25.

52. A system, comprising:
a fluidic sample receiving area, wherein at least one surface of the fluidic sample receiving area comprises one or more one or more binding moieties that bind at least to receptors on Chimeric Antigen Receptor (CAR)-T cells;
a filter mechanism operably connected, or connectable, to the fluidic sample receiving area and/or to another system component, which filter mechanism is configured to prevent at least some non-CAR-T cells in a fluidic sample from contacting the surface of the fluidic sample receiving area when the fluidic sample flows through the fluidic sample receiving area;
a detector operably connected, or connectable, to the fluidic sample receiving area and/or to another system component, which detector is configured to detect a captured cell population bound to the binding moieties when the fluidic sample flows through the fluidic sample receiving area; and,
a controller operably connected, or connectable, to the fluidic sample receiving area and/or to another system component, which controller comprises, or is configured to communicate with, a trained machine learning model that distinguishes the CAR-T cells from the non-CAR-T cells in the captured cell population when the fluidic sample flows through the fluidic sample receiving area.
